(19) United States
(12) Patent Application Publication
(10) Pub. No.: US 2016/0346437 A1
(43) Pub. Date: Dec. 1, 2016

(54) IMPLANT WITH IMPROVED SURFACE PROPERTIES

(71) Applicant: QVANTEQ AG, Zürich (CH)

(72) Inventors: Arik ZUCKER, Zürich (CH); Stefano BUZZI, Zürich (CH); Armin W. MÄDER, Richterswil (CH); Vincent MILLERET, Zürich (CH); Martin EHRBAR, Wil (CH); Algirdas ZIOGAS, Zürich (CH)

(21) Appl. No.: 15/037,015

(22) PCT Filed: Nov. 12, 2014
(86) PCT No.: PCT/EP2014/074394

§ 371 Date: May 16, 2016

(30) Foreign Application Priority Data
Nov. 14, 2013 (CH) 01904/13

(51) Int. Cl. A61L 31/02; A61F 2/86; A61L 31/14

(52) U.S. Cl. CPC A61L 31/022; A61L 31/14; A61F 2/86; A61L 2400/18; A61F 2310/00023

(57) ABSTRACT

An implant for vascular implantation in a body is provided with a surface which, in the implanted state, is provided for contact with the body or with a bodily fluid and which is at least for the most part hydrated. The surface is advantageously provided as a bare metal surface of a chromium-containing alloy. The vascular implant is used to regulate an adsorption of proteins on the surface of the implant, in respect of the type, quantity and/or conformation of certain proteins, by a defined surface which is at least for the most part hydrated.

The present invention relates to an implant for vascular implantation in a body, in particular a vascular prosthesis e.g. in the form of a stent and a use of the implant for regulating an adsorption of proteins on a surface of the implant when implanted.

Implants, such as e.g. stents inserted into blood vessels, entail certain risks for the patient. Among other things, inflammation reactions can arise and another stenosis in the blood vessels can occur e.g. through thrombosis formation on the surface of the implant or through a neointimal hyperplasia. For example impurities on the surface of the implant, which can arise through usual handling and cleaning of the implant or during transfer of the implant, can influence the reaction of the body to the implant. Complications can be triggered through the adsorption of proteins on the surface of the implant as soon as the latter comes into contact with the body or respectively with blood. The quantity and type of adhering proteins determines the further biological reactions between the body and the implant. The adsorption of certain blood components is thereby promoted or decreased, and their effects activated or inhibited. This interaction between implant and body is decisive for the success or failure of the growing together of the implant in the body.

The successful growing together of an implant thus depends on the characteristics and the condition of the surface of the implant. Known from the state of the art are implants with diverse surface coatings, whereby the individual coatings are supposed to support and influence in one way or another the growing together of the implant.

Moreover known from US 2008/0086198 A1 is e.g. a stent with a nanoporous surface layer, which is supposed to improve the growing together of the stent and its re-endothelialization and decrease inflammation and intimal proliferation. The nanoporous surface layer can thereby be provided with one or more therapeutic active ingredients. Experimental results disclosed in US 2008/0086198 A1 for stents with a controllable elution show a lesser restenosis compared with stents with bare metal surface (bare metal stents). With a stent with simple metal surface a chronic irritation of the tissue surrounding the stent is suspected.

Shown in EP 1254673 B1 is a stent, the surface of which is provided in such a way that a recognition of the stent as foreign body is minimized. For this purpose the surface structure of the stent is supposed to mimic the surface structure of the body's own cells. This is achieved by microstructures, spaced apart from one another, on the stent surface which have an extension in the micrometer range. It was discovered that stents of this kind exhibit an improved immunotolerance compared with stents with a smooth or generally rough surface. The growing together of the stent can be further improved in that material is used with a positive surface charge in the range of 0.03 to 0.05 N/m. The adhesion of fibrinogen on the stent surface is thereby reduced. This is supposed to lead to a diminished inflammatory response and thereby decrease the immune reaction.

Implants with coated surfaces or with surfaces provided with structures or a defined roughness are costly in manufacture. Furthermore such surfaces make it difficult to clean the surface and keep it clean during handling in the manufacturing, storing and implantation process. Moreover also with implants of this kind in some cases a renewed restenosis or other complications arise.

It is an object of the invention to provide an implant that reduces complications in the use of the implant in the body, in particular improves a desired growing together of the implant in the body and prevents a restenosis, that makes possible a simple manufacture and handling of the implant, ensures a functioning of the implant in the body in the long term and permits a high level of purification of the implant surface. Furthermore it is an object of the invention to improve an adsorption of proteins on the surface of the implant relating to tolerance of the implant and a successful implantation.

This object is achieved by the invention by means of the implant according to claim 1 and a use of the implant according to claim 14. Advantageous embodiments and preferred examples are described in the dependent claims.

According to the invention an implant is provided for vascular implantation in a body. The implant has a surface, which is provided for contact with the body or a bodily fluid in the implanted state and which is at least for the most part hydrated. The surface thus has a hydration which covers at least the major portion of the surface. Preferably the surface is completely covered with the hydration. The invention encompasses implants of any kind, in particular implants which come into contact with bodily fluids and are employed in the area of fluid dynamics of the body. In particular an implant with the features according to the present invention can be advantageously designed as vascular prosthesis, such as e.g. stents, grafts, heart valves, elements of cardiac pacemakers, etc. The invention relates in particular to cardiovascular implants, which are inserted into soft tissue of the body, such as, for example, stents. In contrast to bone implants, implants of this kind are not supposed to absorb or soak up bodily fluid, such as blood. Stents are, as a rule, of tubular design and are constructed from a multiplicity of crosspieces which form together a kind of mesh. The surface of a stent is formed by the surface of the crosspieces, respectively of the mesh.

Initially the implant is in a first state in which there is no hydration or only a minimal hydration of the surface. The surface is then subjected to a hydrating treatment so that the surface in a second state, in which the implant is inserted into the body or a body lumen, is at least for the most part hydrated. The characteristics of the implant surface in the first state can correspond to the features of a starting material, from which the implant is produced. The first state can also be viewed as the state of a conventionally produced implant provided for implantation. The first state can thus be seen as the starting state of the implant, in which the implant is present e.g. after shaping or a first cleaning. In the starting state the implant can also be already mounted in, or on, an insertion system.

The implant surface is preferably completely hydrated at the time of implantation. This encompasses the inner and outer surface, but also the lateral surfaces, as they exist in the case of a stent, for example, owing to its mesh form. The hydration can be achieved through hydroxide groups which are bound on the surface on which groups water molecules are adsorbed. The hydration preferably comprises at least one water monolayer. A multiplicity of monolayers can also be layered on top of one another. The water monolayer is strongly bound to the hydroxide groups of the surface through hydrogen bridges (H bridges). Further water molecules can be bound more weakly to the water monolayer through dipole-dipole bonds and/or through van der Waals forces and/or through H bridges and thus form further molecule layers.

Surfaces can be contaminated by the surrounding atmosphere by the substances contained therein, such as e.g. hydrocarbons, etc. With the implant according to the invention this contamination should be kept as minimal as possible or even eliminated completely, and, to be precise, on the hydrated surface itself, on the overlying water monolayer and on the further water layers possibly situated over the water monolayer. Contamination should likewise be minimized or even eliminated completely within the aforementioned water layers. A suitable inert covering or packaging, for example, offers a possible protection against such contamination. For maintaining the hydration of the implant surface, a handling and storage of the implant can take place as described in the parallel patent application of the applicant (Application No. CH 00048/12).

In a preferred embodiment, the metal surface for the hydration can have in a first state a first surface charge and can assume, through a surface treatment, a second state with a second surface charge which is a lower positive surface charge or a higher negative surface charge compared with the first surface charge. A bare metal surface with such a surface charge promotes a complete hydration of the surface. A surface with such a surface charge even without an at least approximate hydration can have a positive effect on the growing together of the implant. The applicant therefore reserves the right to direct an own patent application thereto, the contents of which, or respectively the features of the implant surface, being referred to in their entirety for elucidation of embodiments of the present invention.

Hence the surface in the second state, in which the implant is inserted into the body or a body lumen, has overall a more negative surface charge than in the first state. The second surface charge of the surface is preferably negative. This can be achieved through the surface treatment even when the surface charge in the first state has a positive value.

The use of an implant according to the present invention is foreseen for regulating an adsorption of proteins on the surface of the implant in terms of type, quantity and/or conformation of certain proteins by means of a defined surface, which is at least for the most part hydrated. The defined state can also have a defined surface charge and/or a defined predetermined composition of an oxide layer of the surface. The defined state is determined according to a desired regulation of the protein adsorption. Hence for different requirements for protein adsorption differing defined conditions can be established which are each attained by a suitable surface treatment.

Through an implant according to the present invention the quantity of proteins and other elements adhering on the surface during an implantation of the implant can be changed. For example, undesired proteins can be reduced and desired proteins settled in an increased way. More neutrophils can be settled on the implant surface, which release cathelicidin and thus are responsible for a reduction of restenosis. The adsorption of thrombocytes can be decreased. Thus the risk of complications with implantation of an implant is significantly reduced and the growing-together behavior of the implant is improved. Complications from breaking or chipping of coatings on the implant, as is known from the state of the art, are excluded.

Good results have been obtained with implants according to the invention in which the second surface charge is more negative than the first surface charge by at least 10%, preferably by 20% or more. A zeta potential value of the surface in the second state should be below the zeta potential value of the first state. With a pH value of about 7.4, which corresponds to the pH value of blood, a zeta potential value of less than −60 mV, in particular less than −70 mV, is advantageous. The zeta potential can serve e.g. to determine a defined state for the implant surface. The said potential values relate to a determination procedure by means of electrokinetic analysis. With the use of other determination procedures, the indications for potential values may possibly have to be adapted according to the procedural standards.

Furthermore the surface of the implant can be characterized by the isoelectric point on the surface. The isoelectric point is defined as the pH value at which the surface charge is equal to zero. According to the invention, with the surface in the second state there is an isoelectric point which is lower than in the first state of the surface. For example, the isoelectric point with a hydrated metal surface is below 5.0. Thus the isoelectric point can serve to determine a defined state of the surface.

The surface treatment for creating the hydrated implant surface can be provided e.g. through a cleaning treatment and a subsequent storage in a treatment solution. In particular the implant surface can be stored in a neutral or slightly acidic, aqueous solution, for example in a NaCl solution or WFI water (water for injection). For cleaning, the implant surface can e.g. be subjected to a plasma treatment. With metal bare surfaces an oxidation treatment with a subsequent storage in treatment solution is particularly suitable. For creation of a negative surface charge, the implant surface can be subjected to a surface charge reduction treatment, such as e.g. a plasma treatment.

The implant preferably consists of metal or a metal alloy, in particular an alloy containing chrome, such as a cobalt chrome alloy or a platinum chrome alloy, or consists of nitinol. Stainless steel can also be used. Such materials and their properties are well known for use with implants. Especially preferably the implant has a bare metal surface. No coating steps are therefore necessary such as are known e.g. for the coating of medicaments or the like. The surface also does not need to be subsequently treated for producing a particular surface structure. Furthermore a bare surface facilitates the cleaning or purification and thus makes possible highly pure implant surfaces. Provided in a particularly preferred way is a hydrophilic surface. The hydrophilicity can be generated or increased e.g. at the same time with the surface charge reduction treatment or a cleaning treatment. Alternatively a second surface charge and a hydration can also be provided with an implant with a medicinal coating.

The metals or metal alloys used for the implant according to the invention have metal surfaces which have an oxide layer in the outermost position of their metal structure. The oxide layer is 2-3 nm thick and has oxides in accordance with the metal used. A cobalt chrome surface has e.g. a proportion of about ⅔ Cr₂O₃ oxide. With an implant according to the present invention the surface in the second state advantageously has an oxide layer having changed quantities of oxides compared with the oxide layer in the first state, i.e. compared with the starting state. It is also possible for the oxide layer in the second state to have a changed thickness, be preferably thicker, compared with the first state. In the case of an implant of cobalt chrome, the oxide layer of the surface in the second state relative to the first state can have an increased amount of chromium oxide and/or a decreased amount of cobalt oxide and nickel oxide. In the case of a nitinol implant, a reduced quantity of nickel oxide or an elimination of nickel oxide can be achieved. Thus a defined surface charge can be produced on the implant surface with a predetermined composition of different oxides in the oxide layer. With a metal surface for implants, chrome alloys are especially suitable for a selective change of the oxide layer. Used for a surface with a hydration are preferably chrome alloys with at least 5% chrome. Empirical studies have determined that owing to the high affinity of chrome for oxygen, such chrome alloys have an increased proportion of chromium oxide on the surface compared with the chromium content of the alloy itself. The chromium oxide is accordingly preferably adsorbed outside on the alloy and in increased concentration. According to the invention, an implant is preferably used which consists of a chromium-containing alloy, whereby the surface has an oxide layer in which at least 30% of the oxide consists of chromium oxide, preferably at least 50%. According to the invention the chromium oxide reacts with the water molecules and results in chromium hydroxide, which is hydrated with at least one bound water monolayer. With such a proportion of hydrated chromium hydroxides it can be ensured that the implant surface is at least for the most part hydrated.

The conformation of proteins adsorbed on an implant surface likewise has an influence on the adhesion of neutrophils and thrombocytes and thus on the growing-together behavior of an implant. Proteins are complex copolymers, whose three-dimensional structure is composed of several levels. Involved in the structural composition can be amino acid sequences, different α-helix and β-sheet structures, the common structure of a multiplicity of polypeptides and the like. Understood as natural conformation is a conformation of proteins which the proteins assume when no outside influences take effect on the three-dimensional structure of the proteins and influence these proteins. To be designated as an almost natural, or respectively natural-like conformation should be a conformation in which slight changes in the protein structure exist, but these changes have no influence or a negligible influence on the function and effect of the protein. With proteins there are different regions, e.g. positively or negatively charged regions, hydrophilic and hydrophobic regions, which, depending upon spatial organization of the proteins, are exposed and can carry out specific biological functions. Through adsorption on a surface the protein conformation changes. Generally a protein has e.g. on a hydrophobic surface a greatly denatured conformation, while there exists on the hydrophilic surface a less denatured conformation. The hydrophilic components of the proteins in the natural conformation usually lie outside and the hydrophobic components usually lie inside and are accessible for the hydrophobic surface only through a major conformation change. Information about the protein conformation can be gained through a measurement of the behavior of α-helix and β-sheets or through a measurement of specific amino acids on the protein surface.

With the present invention it was surprisingly discovered that e.g. fibrinogen can be settled on an implant surface according to the invention at least approximately in its natural, or respectively natural-like, conformation, as has been confirmed by above-mentioned observations. The effect of fibrinogen on an implant surface according to the invention can be improved, since fibrinogen is adsorbed primarily in an advantageous conformation. In contrast thereto, fibrinogen on an implant surface in the starting state of a metal surface is adsorbed in a denatured state, whereby a negative influence on the growing together of an implant results. In a denatured state fibrinogen has a changed three-dimensional structure and a changed spatial distribution of different fibrinogen regions than in natural state. A natural conformation also with other proteins promotes a positive growing together of the implant.

During the implantation in a body lumen, the body's own defense or resistance can recognize the difference between natural and denatured protein, in particular of fibrinogen, so that denatured protein is identified as foreign body and an adverse reaction is triggered. Fibrinogen and other proteins in a natural conformation can be beneficial for a healthy growing together behavior of an implant, whereas e.g. fibrinogen in a denatured conformation is detrimental to the growing-together behavior. The mere amount of fibrinogen is therefore less decisive for the growing together of the implant.

The applicant therefore reserves the right to direct an own patent application to an implant for vascular implantation into a body with a surface which is provided for contact with the body or a bodily fluid in implanted state, the surface having a layer of proteins, in particular of fibrinogen, in an at least almost natural, or respectively natural-like, conformation. The layer of proteins in an almost natural conformation is advantageously provided on a bare metal surface of the implant. Furthermore the layer is advantageously provided on a hydrophilic surface of the implant. The remarks about the features and the advantages of a layer of proteins in an at least approximately natural, or respectively natural-like, conformation from such a patent application are fully incorporated in the scope of the present patent application in order to supplement and support the remarks concerning the present invention.

With use of the implant the amount of adsorbed proteins can vary in the defined second state of the surface compared with the starting state of the implant surface. For example, the absolute amount of adsorbed proteins can be decreased and/or certain kinds of proteins can be adsorbed in an increased way and other kinds of proteins adsorbed in a decreased way. Thus the risk can be reduced of undesired deposits of proteins. The type of adhering proteins can thus be regulated in that a suitable defined second state is generated with the hydration and for example different oxides in the oxide layer or different surface charge. Through the production of an implant with a hydrated surface the adsorption of the proteins can be influenced. Less macroglobulin and/or apolipoprotein A can adhere on the surface and more apolipoprotein E, kininogen and/or plasminogen can be adsorbed. Above and beyond this, the conformation of proteins on the surface can be regulated. For example, fibrinogen can be settled on the implant surface in a way corresponding to its natural conformation, as explained above. Its natural effectiveness is thereby preserved and the deposit of neutrophils promoted.

Embodiment examples and experimental results for implants according to the invention will be explained in the following with reference to figures, which are not to be interpreted in a limiting way. Features and interrelationships emerging from the figures should be viewed as belonging to the disclosure of the invention individually and in any combination. In the figures:

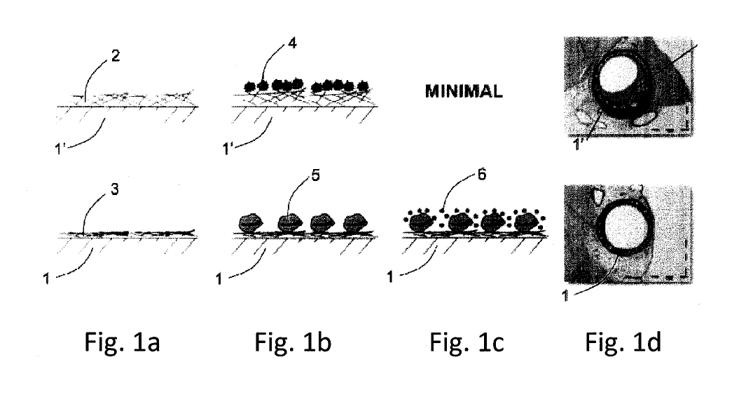

FIG. 1 shows a schematic course of the growing together of a conventional bare metal stent (above) and a bare metal stent according to the invention with an increased negative surface charge according to the invention (below),

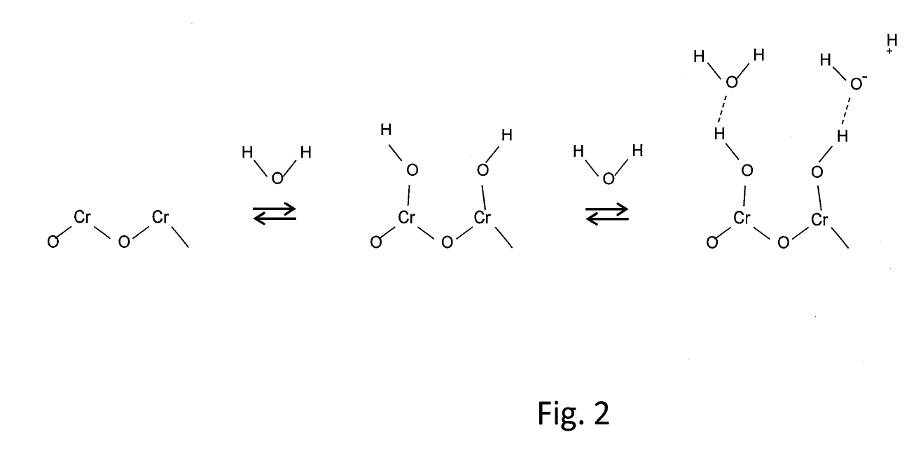

FIG. 2 shows a diagram of the chemical process in a hydration of a metal surface with chromium oxide,

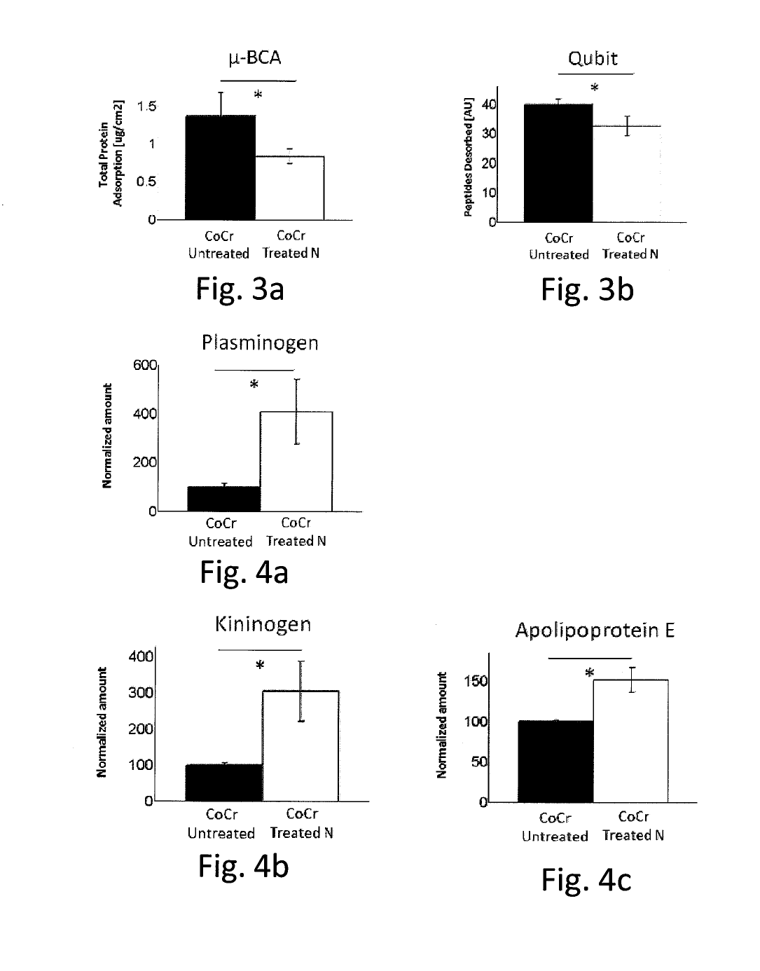

FIG. 3a shows a diagram of the quantity of adsorbed proteins on an implant metal sample with a cobalt chrome surface with a first surface charge and a second surface charge from a measurement by means of μ-BCA method,

FIG. 3b shows a diagram of the quantity of adsorbed proteins on an implant metal sample with a cobalt chrome surface with a first surface charge and a second surface charge from a measurement by means of Qubit method,

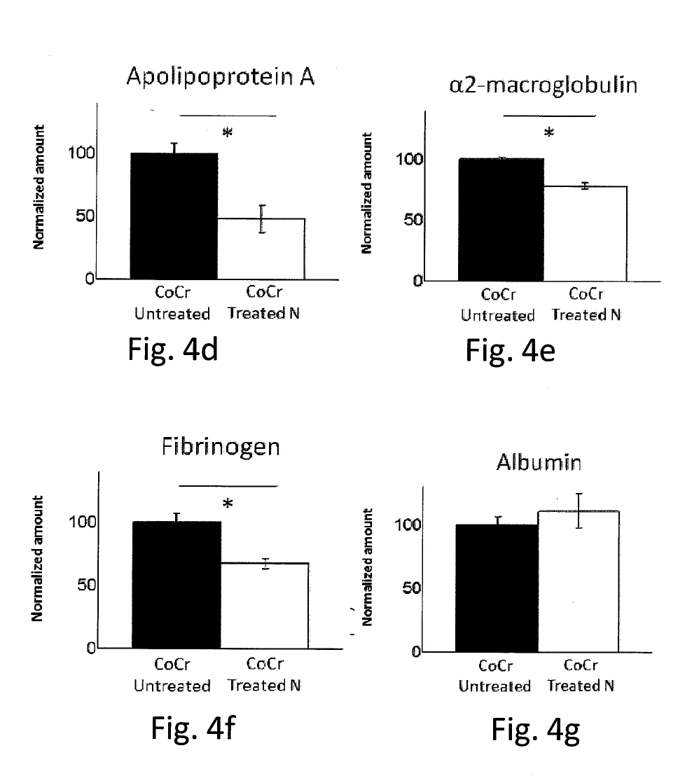

FIGS. 4a-4g show diagrams of the protein adsorption of an implant metal sample with a cobalt chrome surface with a first surface charge and a second surface charge for the proteins plasminogen, kininogen, apolipoprotein E, apolipoprotein A, α2-makroglobulin, fibrinogen and albumin,

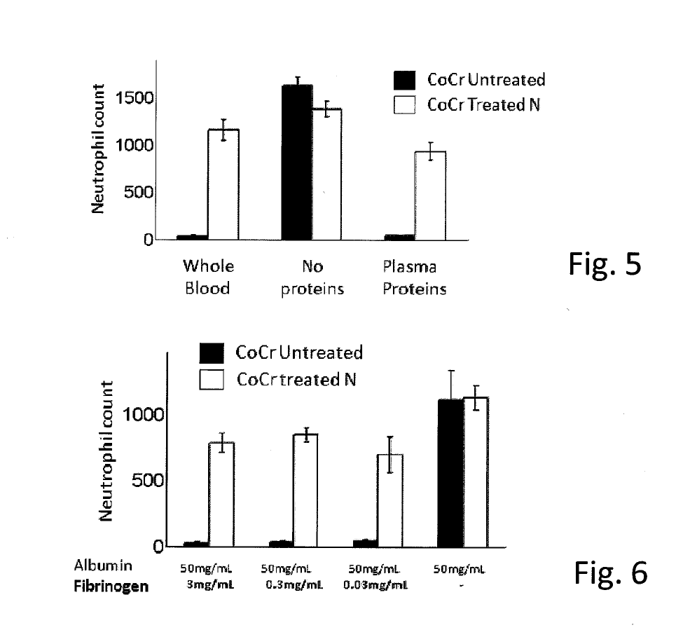

FIG. 5 shows a diagram of a number of neutrophils on sample surfaces with a first surface charge and a second surface charge in different environments, and

FIG. 6 shows a diagram of the correlation of a fibrinogen concentration with respect to the adsorption of neutrophils.

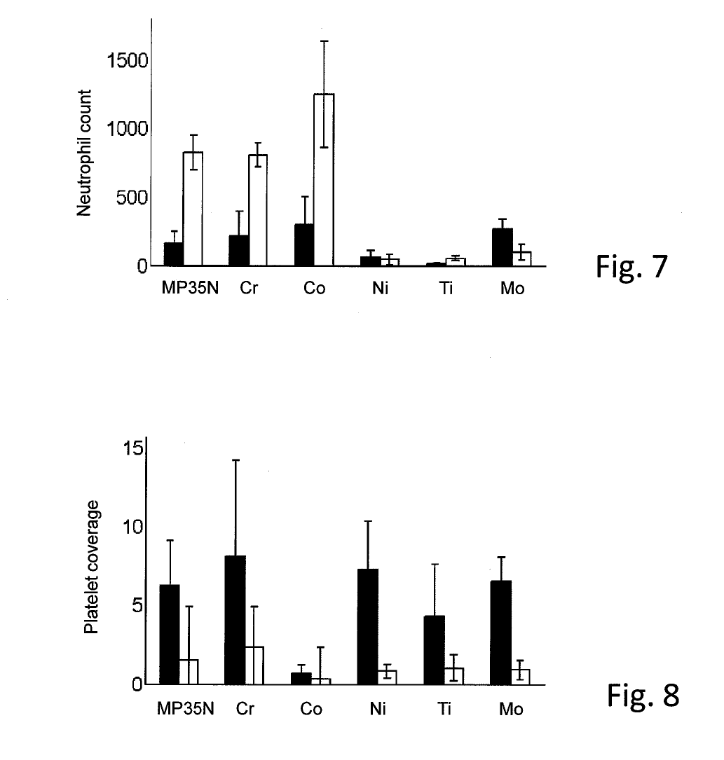

FIG. 7 shows a diagram of a number of neutrophils on sample surfaces with an untreated surface and a hydrated surface for different sample surfaces, and

FIG. 8 shows a diagram of a number of thrombocytes on sample surfaces with an untreated surface and a hydrated surface for different sample surfaces.

Various experiments were conducted and different measuring methods used in order to study the significance of the improved growing together of an implant for vascular implantation according to the present invention. It was thereby clearly determined that an implant with an at least for the most part hydrated surface promotes a growing together of the implant without complications, compared with an implant surface without or with only minimal hydration, or respectively an implant surface without a hydration treatment.

Used as vascular implant was a stent with bare metal surface as produced e.g. in the state of the art and used as vascular prosthesis. The outer surface of the stent is foreseen to abut a vascular wall of a body. The surfaces of the stent come into contact with the blood in the vessel. Further used were metal samples e.g. in the form of disks for carrying out surface measurements. The metal samples consist of a metal or metal alloy as is also used for a vascular implant, respectively the stent. Thus the metal surfaces of the samples are equivalent to surfaces of stents provided for implantation. Cobalt chrome, platinum chrome and nitinol are studied. In principle other metals or metal alloys with comparable features could also be used for an implant according to the invention.

Used in the subsequently described measurements were the following metal samples: a cobalt chromium alloy MP35N (ASTM F562) consisting of about 34 wt % cobalt, about 35 wt % nickel, about 20 wt % chrome, about 10 wt % molybdenum and less than 1 wt % of titanium and iron and a cobalt chrome alloy L605 (ASTM F90) consisting of about 51 wt % cobalt, about 20 wt % chrome, about 15 wt % tungsten, about 10 wt % nickel, less than 3 wt % iron, about 1.5 wt % manganese and less than 1 wt % silicon.

The following measurement methods and measuring devices were used: X-ray photoelectron spectroscopy (XPS measurement) with a Kratos AXIS NOVA™ device on 12 different samples and zeta potential measurement with a SurPASS™ electrokinetic analyzer with variable pH value on two different samples.

The studied stents and the metal samples are first in a first state without, or with only minimal, hydration, which corresponds to the starting state. The starting state is e.g. that of a stent as used in a conventional way for implantation. The stent is thus ready made in the starting state and is ready for implantation in the sense of the state of the art. To create the second state with an at least for the most part hydrated surface, the stent and the metal samples are subjected to a surface treatment. Such a surface treatment to produce the hydration can be e.g. a bath in a previously mentioned solution.

Furthermore a surface treatment to change the surface charge can take place, e.g. through an oxidation treatment in the form of a plasma treatment and/or a bath in a previously mentioned aqueous solution. The plasma treatment leads to an oxidation and removal of hydrocarbon. For the plasma different gases can be used, as they are known from the state of the art. For example, an oxygen plasma is used. The bath can have a predetermined pH value which is coordinated with the material of the metal sample. For example, an alkaline solution is used. Used to reduce the surface charge on the surface is, for example, an argon plasma, which does not act in an oxidizing way, in combination with a bath in an aqueous NaCl solution, which acts in an oxidizing way. The treated surface has uniform surface characteristics with a second lower surface charge and a hydration in the sense of the invention.

To maintain the hydration of the implant surface a handling and storage of the implant can take place as are described in the parallel patent application of the applicant (application number CH 00048/12). This application is fully incorporated by reference into the disclosure of the invention since it discloses in what way stent surfaces with defined surface features can be maintained until implantation. The second state of the implant surface can be maintained by providing a stent inside a flow of a defined medium in a cover.

The stent can also be subjected to a surface treatment when it is already put in, or on, an insertion system for inserting the stent into the body or a body lumen, or can be inserted into such a system after the treatment. Care must thereby be taken to ensure that the surface charge of the second state is preserved.

Shown in FIGS. 1a to 1c is the course of the growing together of a conventional metal stent 1′ with bare surface in a first state (above) and a metal stent 1 according to the invention with bare surface in a second state with an increased negative surface charge (below). FIG. 1d shows for the stents 1 and 1′ the growing together of the stents in a coronary artery of a pig after 30 days.

In FIG. 1a the stent is placed at the site of the implantation and the surfaces are exposed to blood. With the conventional stent 1′ (FIG. 1a, above) there takes place initially a depositing of proteins, which prevent the adherence as well as the functionality of neutrophils, so-called neutrophil inhibitors 2 (α²-makroglobulin, apolipoprotein A). With stent 1 with increased negative surface charge (FIG. 1a, below) the neutrophil inhibitors are greatly reduced and at the same time proteins are deposited that prevent the adherence of thrombocytes (high molecular weight kininogen—HMWK), and also proteins are deposited that promote the adherence of neutrophils on the stent surface (e.g. plasminogen, fibrinogen in natural or at least natural-like conformation), so-called neutrophil promotors 3. Accordingly, with the stent 1′ in the first state (FIG. 1b, above), primarily thrombocytes 4 are then settled on the neutrophil inhibitors 2, which are basically undesired. With the stent 1 with increased negative surface charge (FIG. 1b, below), on the other hand, neutrophils 5 from the blood of the patient are settled on the neutrophil promoters 3 and activated, while thrombocytes are repelled. The activated neutrophils 5 release the protein cathelicidin (LL37) 6 on the stent surface, cf. FIG. 1c below. The process of growing together can thereby be positively supported without a medicinal coating having to be applied to the metal surface for this purpose or without delivery of a medicine being necessary. In the case of the stent 1′ in the first state cathelicidin was found only in minimal amounts. The studies have shown that the stent 1 in the second state accumulates two to three times more cathelicidin than the conventional stent.

FIG. 1d shows for the stents 1 and 1′ the growing together of the stents in a coronary artery of a pig after 30 days. The stent 1 with a hydrated surface shows a uniform growing-together behavior with a wide open inner lumen (see FIG. 1d, below). The stent 1′ in the starting state however shows a growing together with a renewed narrowing of the passage (see FIG. 1d, above). In summary, it can be observed: The surface of the stent 1 having an at least for the most part hydrated surface compared with conventional stents supports and promotes those bioactive processes which lead to a healthy and desired growing together of the stent 1. Undesired processes, on the other hand, are curtailed or inhibited.

FIG. 2 shows the conditions in the case of hydration of an implant surface with chromium oxide in the outer oxide layer. Shown on the left is the chromium oxide in a state without hydrating. Owing to their high affinity for oxygen, the chrome atoms have bound to oxygen atoms. Starting from this state, through a chemisorption of water molecules, the chromium oxide turns into chromium hydroxide in which the hydroxy groups are bound by a strong bond on the surface (FIG. 2, in the middle). Through water physisorption, a further state is obtained (FIG. 2, on the right) with a physisorbed water layer, in which the water can be partially dissociated. The water molecules are thereby bound to the hydroxy groups through a hydrogen bridge bond. In this state the implant surface has a water monolayer, as is preferably foreseen for the hydration according to the invention. Further water molecules can be adsorbed on this water monolayer, e.g. through a water bath, and thus a thicker layer of water achieved. These further water molecules are bound more weakly, for example by van der Waals or dipole-dipole forces. The physisorption of water molecules can be supported through a suitable pretreatment, e.g. a cleaning or purification and in particular through a reduction of the surface charge, so that an at least for the most part hydrated implant surface can be reliably produced.

Experimental studies of an oxide layer on the surface of MP35N and L605 samples have shown that in the starting state the oxide layer has a thickness of 2-3 nm. In the measurement of MP35N samples using the electrokinetic analyzer (XPS measurement) the following was determined. With a first MP35N sample in the starting state, the oxide layer is composed essentially of about 66% Cr₂O₃ (Cr(III)) oxide, about 10% Co oxide, about 10% Mo oxide, about 9% Ni oxide, about 5% Ti oxide. A second MP35N sample was subjected to storage in a neutral solution following an oxidation treatment and is thus in a second state according to the invention. In the case of the second MP35N sample the oxide layer likewise has a thickness von 2-3 nm and consists of 75% Cr₂O₃ (Cr(III)) oxide, about 7% Co oxide, about 8% Mo oxide, about 7% Ni oxide and about 4% Ti oxide. In measuring the L605 samples comparable results were obtained. Only molybdenum is substituted by tungsten and less nickel is measured, which is compensated by cobalt, as corresponds to the different ratios of the metals in the different alloys. A greater quantity of chrome oxide and a lesser quantity of cobalt oxide and nickel oxide were measured. This means that in the second state with an increased negative surface charge the amount of chrome oxide is higher and the amount of cobalt oxide and nickel oxide is lower than in the first state. Above and beyond this a hydration forms on the surface, the water molecules binding e.g. to the chrome ions, as explained previously.

As previously mentioned, through the type of surface treatment, that is e.g. cleaning through plasma treatment and wet storage in solutions, on the one hand the surface charge can be changed to a more negative value, and, on the other hand, the composition of the oxide layer can be influenced and thus regulated.

The zeta potential and the surface charge were determined for the two metal samples, made of different cobalt chrome alloys, used in the studies. Measured was the zeta potential at a pH value of 7.4 in diluted KCl solution, as corresponds to the pH condition in blood. In the second state after the surface treatment, there was in the case of the MP35N sample a zeta potential of about −95 mV. For the L605 sample a zeta potential of −80 mV was measured. This corresponds to a more negative surface charge for both samples after the surface treatment. Both treated samples have an isoelectric point under 5.0. The zeta potential was determined by means of electrokinetic analysis.

Shown in FIGS. 3a and 3b are results from a determination of the total quantity of proteins adsorbed on the surfaces. FIG. 3a shows the result of a μ-BCA measurement in which the effect of the protein-copper-chelate formation and the reduction of the copper with bicinchoninic acid (BCA) to a colored solution product is made use of for a fluorescence measurement. FIG. 3b shows the result of a Qubit measurement in which the proteins adhering to the surface are desorbed and are provided with a marker for a fluorescence analysis. With both measurement methods a significant reduction in protein adsorption was shown.

Shown in FIG. 3a is the total adsorption of proteins for a metal sample without or with minimal hydration (first state, left bar) and for the metal sample with a hydrated surface (second state, right bar). In the first state between 1.2 and 1.7 μg/cm² of proteins are adsorbed on the metal surface. In contrast, in the second state between 0.7 and 0.9 μg/cm² of proteins are adsorbed. Shown in FIG. 3b is the total adsorption of proteins for a metal sample without or with minimal hydration (first state, left bar) and for the metal sample with a hydrated surface (second state, right bar). In the first state between 36 and 40 arbitrary units of proteins are measured on the surface. In contrast, in the second state between 30 and 34 arbitrary units of proteins are measured.

Shown in FIGS. 4a to 4g are diagrams of measurements of the protein adsorption of an implant with a cobalt chrome surface without or with minimal hydration (first state) and with an at least predominantly hydrated surface (second state) for the proteins plasminogen (FIG. 4a), kininogen (FIG. 4b), apolipoprotein E (FIG. 4c), apolipoprotein A (FIG. 4d), α2-macroglobulin (FIG. 4e), fibrinogen (FIG. 4f) and albumin (FIG. 4g). The metal samples correspond to the material of an implant according to the invention, and have a bare cobalt chrome surface. In the first state the surface is measured without further treatment steps, i.e. in the starting state. In the second state the surface was subjected to a hydrating treatment, as previously described, and thus has an at least for the most part hydrated surface according to the invention. The zeta potential in the first state is at about −55 mV and in the second state at about −95 mV. The metal samples were incubated for measurement of the protein adsorption in blood. For this purpose the samples were placed in dishes with fresh blood and incubated for two hours at 37° C. and static conditions. Then the samples were measured with the previously mentioned method. The results are presented in FIGS. 4a to 4g in such a way that the adsorption on the untreated surface is normalized to 100. The deviation therefrom, which occurs with a treated surface, thereby becomes clearly visible.

Shown in FIG. 4a is the quantity of plasminogen on the sample surface in the first state (left bar) and in the second state (right bar). The adsorption in the first state is normalized to 100+/−10. In contrast, in the second state a value of 400+/−150 is reached. Thus in the second state there is a significantly greater amount of plasminogen, which, among other things, is responsible for the adsorption of neutrophils.

Shown in FIG. 4b is the amount of kininogen on the stent surface in the first state (left bar) and in the second state (right bar). The adsorption in the first state is normalized to 100+/−5. In contrast, in the second state a value of 300+/−80 is reached. Shown in FIG. 4c is the amount of apolipoprotein E on the stent surface in the first state (left bar) and in the second state (right bar). The adsorption in the first state is normalized to 100+/−3. In the second state a value of 150+/−30 is reached. Known about kininogen and apolipoprotein E is that they prevent the aggregation of thrombocytes. Thus the quantity of thrombocytes on an implanted stent surface can be regulated through the increase of the proteins kininogen and apolipoprotein E on the surface.

Shown in FIG. 4d is the quantity of apolipoprotein A on the stent surface in the first state (left bar) and in the second state (right bar). The adsorption in the first state is normalized to 100+/−10. In the second state, on the other hand, only a value of 50+/−12 is reached. Shown in FIG. 4e is the amount of α2-macroglobulin on the stent surface in the first state (left bar) and in the second state (right bar). The adsorption in the first state is normalized to 100+/−2. In contrast, in the second state only a value of 80+/−3 is reached. Thus in the second state in each case there is a significantly lesser quantity of apolipoprotein A and α2-macroglobulin. Apolipoprotein A and α2-macroglobulin reduce the adsorption of neutrophils and inhibit the function of neutrophils. Apolipoprotein A inhibits moreover cathelicidin (LL-37), which promotes a positive growing together of implants.

Shown in FIG. 4f is the quantity of fibrinogen on the stent surface in the first state (left bar) and in the second state (right bar). The adsorption in the first state is normalized to 100+/−10. In contrast, in the second state only a value of 70+/−5 is reached. Fibrinogen can promote the adsorption of thrombocytes and inhibit neutrophils. It is therefore advantageous to reduce the amount of fibrinogen on the implant surface.

Shown in FIG. 4g is the quantity of albumin on the stent surface in the first state (left bar) and in the second state (right bar). The adsorption in the first state is normalized to 100+/−10. In the second state, on the other hand, a value of 115+/−15 is achieved.

In summary it can be observed that the stent surface in the second state with a hydrated surface compared with the surface charge in the first state has fewer proteins which reduce the quantity and functioning of neutrophils on the surface, and has more proteins which reduce the aggregation of thrombocytes. The results show that the metal surface in the second state is occupied by a lesser quantity of proteins than in the first state. Preferably thrombocyte inhibitors, such as kininogen, and neutrophil promoters, such as plasminogen, are adsorbed. Furthermore fewer neutrophil inhibitors, such as e.g. apolipoprotein A and α2-macroglobulin are settled on the surface. Therefore neutrophils can adhere quickly to an implanted surface and support a successful growing together of the stent.

With a preferred procedure, through a surface charge reduction treatment and/or through an oxidation treatment, a defined second surface charge and/or a defined predetermined composition of the oxide layer, as described above, is produced on the implant surface, which is coordinated with a defined adsorption of predetermined quantities of different proteins on the surface and which supports the hydration of the surface. Through the creation of a defined surface, the adsorption of proteins can be influenced, the adsorption of desired proteins is promoted and the adsorption of undesired proteins is inhibited. Thus a selective protein adhesion on the metal surface takes place. A stent with a hydrated surface according to the invention can reduce the quantity of the proteins fibrinogen, α2-macroglobulin and/or apolipoprotein A being deposited on the implant and increase the quantity of the proteins apolipoprotein E, kininogen and/or plasminogen.

These interrelationships are confirmed by measurements of the quantity of neutrophils on a hydrated surface of a metal sample, which corresponds to a stent surface, and such a surface without or with minimal hydration. In FIG. 5 cobalt chrome samples are incubated in fluids with different proportions of proteins. In principle proteins act as mediators for the settlement of neutrophils, whereby first proteins adhere to the surface and then neutrophils. The left pair of bars shows a measurement in which the metal sample is exposed to regular, human blood. It is shown that the sample without hydration (left bar), accepts only about 8% of the quantity of neutrophils compared with a hydrated surface (right bar). The middle pair of bars in FIG. 5 shows a measurement in which a metal sample was exposed to a fluid with neutrophils, which contained no proteins however, as would normally be the case with a blood fluid. In the hydrated state (right bar) approximately 15% fewer neutrophils are adsorbed than in the first, untreated state (left bar). The right pair of bars shows a measurement in which a metal sample was first incubated in blood plasma. That means that first a depositing of proteins from blood took place and then an adsorption of neutrophils. In the hydrated state (right bar) approximately 20 times more neutrophils are deposited than in the state without or with minimal hydration (left bar). The measurements confirm that with a hydrated metal surface, compared with an untreated metal surface in the starting state, significantly increased numbers of neutrophils are adsorbed, provided that proteins are available which can react with the surface.

Illustrated in FIG. 6, with the example of the protein fibrinogen, is the influence of the presence of this protein on a metal surface state with and without hydration. Comparable measurements are also possible for other proteins. In the series of measurements in each case an untreated metal surface without hydration and a treated, hydrated surface are exposed to a fluid with unchanging proportion of albumin of 50 mg/ml and different proportions of fibrinogen, and the amount of adsorbed neutrophils is measured. With the pair of bars for untreated (left) and treated (right) metal samples the following proportions of fibrinogen were used, seen from left to right: 3 mg/ml, 0.3 mg/ml and 0.03 mg/ml. With all three measurements 15-20 times more neutrophils were adsorbed on the hydrated metal surface than in the case of the untreated metal surface. The measurements confirm that a hydrated surface, compared with the untreated surface, reduces the adsorption of fibrinogen and thereby increases the number of adsorbed neutrophils, which promotes a positive growing-together behavior of an implant. Above and beyond this, the available amount of fibrinogen is not alone decisive, but also its conformation, as previously explained.

The measurements carried out prove the positive effect of an implant surface with a hydrated surface on the growing together of an implant after implantation, as is shown in the in-vivo experiments illustrated in FIGS. 1a to 1d. Through a targeted adjustment of the surface properties on the implant surface the adsorption of proteins on the surface can be regulated. An implant with an at least for the most part hydrated surface compared with conventionally used implants thus reduces the risk of a restenosis or other complications with the implantation.

FIGS. 7 and 8 show the adsorption of neutrophils (FIG. 7) and thrombocytes (FIG. 8) on a multiplicity of different metal surfaces. Measurements were carried out on the surface of an MP35N metal sample, as described above, and on surfaces of pure metal. The pure metal surfaces comprise chromium, cobalt, nickel, titanium and molybdenum. These samples were produced by glass plates being coated with the respective pure metal (chromium, cobalt, nickel, titanium, molybdenum) by means of PVD method (Physical Vapor Deposition), for example by vaporization. In each case a sample with no or only a minimal hydration (left bar) and an at least for the most part hydrated sample (right bar) were incubated in regular, human blood. Then measured by means of fluorescence microscopy was the number of neutrophils on the surfaces (FIG. 7) or respectively the covering of the surfaces with thrombocytes (FIG. 8).

As shown in FIG. 7, in the case of the MP35N sample with minimal hydration, absorbed <sic. adsorbed> are only about 22% of the quantity of neutrophils on the hydrated MP35N sample. A comparable result was obtained for a pure chrome metal sample. The pair of bars for the pure cobalt metal sample shows that on a hydrated surface significantly more neutrophils are settled than on a hydrated chrome metal surface or MP35N surface. With the non-hydrated cobalt metal sample (left) adsorbed are only about 23% of the quantity of neutrophils on the hydrated cobalt metal sample. The nickel, titanium and molybdenum samples show only a minimal neutrophil adsorption. With the nickel metal sample, in the case of the non-hydrated sample slightly more neutrophils are adsorbed than with the hydrated sample. The titanium sample shows the most minimal value for the neutrophil adsorption; the neutrophil adsorption with a non-hydrated sample is thereby almost negligible. With the molybdenum sample, in the case of the non-hydrated sample, about 100% more neutrophils are adsorbed than with the hydrated sample, however overall significantly fewer than with an at least for the most part hydrated surface of a chromium or cobalt metal sample. The measurements show that the hydration in the case of an MP35N alloy, in the case of a chromium metal sample and a cobalt metal sample can significantly increase the adsorption of neutrophils and thereby support a desired growing together of the implant.

As shown in FIG. 8, in the case of the MP35N sample with minimal hydration, about four times more thrombocytes are adsorbed than on the hydrated MP35N sample. A comparable result is obtained for a pure chromium metal sample, whereby with the two chromium metal samples somewhat more thrombocytes are adsorbed than with the MP35N sample. In the case of the cobalt metal sample, in general only few thrombocytes are adsorbed. With the non-hydrated cobalt metal sample (links) adsorbed are only about 100% more thrombocytes than on the hydrated cobalt metal sample. With the nickel, titanium and molybdenum samples, the hydrated samples show a negligible number of thrombocytes, while with the non-hydrated samples the adsorption of thrombocytes is clearly verifiable and lies in the range of the MP35N and chrome samples. The measurements show for all samples a clearly reduced adsorption with hydrated surface compared with a non-hydrated surface. Hence the hydration of the surface reduces the adsorption of thrombocytes regardless of the type of metal and can support the growing together behavior in a further positive way.

In summary, it can be seen from FIG. 7 and FIG. 8 that comparable values can be measured for the MP35N sample and for the pure chrome sample, and, to be precise, both with no or only minimal hydration (left bar), and also for the case of an at least for the most part hydrated surface (right bar). From this it can be concluded that with the MP35N sample primarily chromium oxides in the oxide layer are responsible for the adsorption both of neutrophils as well as of thrombocytes. Therefore, according to the invention, the use of chromium-containing alloys is preferred for the production of the implants with a hydrated surface.

### LIST OF REFERENCE NUMERALS

1, 1′ implant, stent
2 neutrophil inhibitors
3 neutrophil promoters
4 thrombocytes
5 neutrophils
6 cathelicidin

1. An implant, for vascular implantation into a body, with a surface which is provided for contact with the body or a bodily fluid in implanted state, wherein the surface is at least for the most part hydrated.

2. The implant according to claim 1, wherein the surface is completely hydrated.

3. The implant according to claim 1, wherein for hydration hydroxide groups are bound on the surface on which groups water molecules are adsorbed.

4. The implant according to claim 1, wherein the hydration comprises at least one water monolayer on the surface.

5. The implant according to claim 1, wherein the hydration comprises a layer with physisorbed water.

6. The implant according to claim 1, wherein the surface is a bare metal surface.

7. The implant according to claim 1, wherein the implant is composed of metal or a metal alloy, in particular a chromium-containing alloy or of nitinol.

8. The implant according to claim 7, wherein the alloy has at least 5% chrome.

9. The implant according to claim 1, wherein an oxide layer of the surface has at least 30% chromium oxide.

10. The implant according to claim 1, wherein with the surface a defined surface charge and/or a defined predetermined composition of the oxide layer is provided.

11. The implant according to claim 1, wherein the implant is composed of a chromium-containing alloy and the surface has an oxide layer with at least 30% chromium oxide, and the chromium oxide is hydrated.

12. The implant according to claim 1, wherein the surface, in a first state, has a first surface charge and by means of a surface treatment assumes a second state with a second surface charge, the second surface charge being a lower positive surface charge or a higher negative surface charge compared with the first surface charge.

13. The implant according to claim 1, wherein the surface at a pH value of about 7.4 has a zeta potential value of less than −60 mV, in particular less than −70 mV.

14. The implant according to claim 1, wherein the defined second surface charge and/or the defined predetermined composition of the oxide layer is coordinated with a defined adsorption of predetermined quantities of different proteins on the hydration.

15. Use of an implant for vascular implantation according to claim 1 for regulation of an adsorption of proteins on the surface of the implant in terms of type, quantity and/or conformation of certain proteins by means of a defined surface which is at least for the most part hydrated.

16. The implant according to claim 1, wherein the implant is composed of a chromium-containing alloy and the surface has an oxide layer with at least 50% chromium oxide, and the chromium oxide is hydrated.
